(19) United States
(12) Patent Application Publication
(10) Pub. No.: US 2026/0207110 A1
(43) Pub. Date: Jul. 23, 2026

(54) DEVICE FOR PROCESSING CARDIAC ELECTROGRAM SPATIOTEMPORAL DISPERSION SIGNALS

(71) Applicant: SUBSTRATE HD, Marseille (FR)

(72) Inventors: Thomas DEMARCY, Marseille (FR); Léandre GENIN, Marseille (FR)

(73) Assignee: SUBSTRATE HD, Marseille (FR)

(21) Appl. No.: 19/143,214

(22) PCT Filed: Dec. 19, 2023
(86) PCT No.: PCT/FR2023/052063

§ 371 Date: Jun. 25, 2025

(30) Foreign Application Priority Data
Dec. 27, 2022 (FR) FR2214567

(51) Int. Cl. A61B 5/346; G16H 40/63; G16H 50/30

(52) U.S. Cl. CPC A61B 5/346; G16H 40/63; G16H 50/30; A61B 2505/05

(57) ABSTRACT

A device for processing spatiotemporal dispersion signals of electrocardiograms. The device comprising a memory configured to receive spatiotemporal dispersion signals of electrocardiograms associated with a time marker and an electrocardiogram track. The device comprising a calculator configured to receive as input an electrogram track identifier and a time marker, to analyse the spatiotemporal dispersion signal of electrocardiograms associated with this electrogram track identifier, analyse a signal extract comprised between the time marker and the first previous time marker whose spatiotemporal dispersion signal value of electrocardiograms indicates a lack of dispersion, derive from this signal extract a flatness value of the signal, and a duration value derived from the duration of the signal extract, and feed back a track priority value calculated from the flatness value and the duration value. The device comprising a monitor configured to receive the spatiotemporal dispersion signals of electrocardiograms associated with each electrocardiogram track.

### CROSS-REFERENCE TO RELATED APPLICATION(S)

This application is a 35 U.S.C. § 371 National Stage Of International Patent Application No. PCT/FR2023/052063, filed on Dec. 19, 2023 claiming priority to and the benefit of French Patent Application No. FR 2214567, filed on Dec. 27, 2022, each of which is incorporated by reference herein in its entirety for all purposes.

### TECHNICAL FIELD

The invention relates to the field of analysing signals derived from electrocardiograms.

### BACKGROUND

The field of treating atrial fibrillation has made significant progress over the past decade. To treat atrial fibrillation, practitioners operate by inserting catheters provided with a plurality of electrodes. These electrodes are displaced within the heart to measure the electrical signal that propagates therein. The signals obtained are called electrocardiograms. These electrocardiograms are processed in order to help practitioners to detect the zone(s) of the heart that are the cause of atrial fibrillation. Once these zones are identified, the practitioner burns them to make them inactive, which results in the heart restoring normal function and suppressing atrial fibrillation.

Most existing solutions are based on CFAE (“Complex Fractionated Atrial Electrograms”) analysis. The principle is to try to find locations in the atria where the electrograms lose their continuity, i.e. become fractionated.

### SUMMARY OF THE INVENTION

Thea Applicant has developed an electrogram processing that has aroused interest of the scientific community since the publication of very positive clinical studies. These led to the publication of several articles, including that of Seitz et al. “AF Ablation Guided by Spatiotemporal Electrogram Dispersion Without Pulmonary Vein Isolation: A Wholly Patient-Tailored Approach”, Journal of the American College of Cardiology, Volume 69, Issue 3, 24 Jan. 2017, pages 303-321.

This processing is based on the detection of a magnitude of the electrocardiogram signals called dispersion by the Applicant. More specifically, the Applicant has discovered that measuring the spatiotemporal dispersion of electrocardiograms, i.e. based on both the evolution of the electrocardiogram signals of each electrode over time, but also taking account of the electrocardiogram signals of neighbouring electrodes, is particularly effective in order to determine zones of the heart that are at the origin of atrial fibrillation. The measurement of this magnitude has been the subject of a patent issued in many countries in the world, and published in Europe as EP 3, 236, 843.

By extending its work, the Applicant has integrated dispersion into a machine learning tool, in order to determine in near real time the presence of a dispersion within electrocardiograms during a procedure, which makes it possible to alert the practitioner, speed up the ablation procedure as well as the quality of the outcomes obtained while reducing the surgical risks. In addition, the reliability of the procedure is increased and makes it possible to limit the number of patients having to undergo another procedure for the same causes.

This work was incorporated into a software called VX1 which received FDA (“Food and Drug Administration”) approval and CE (European Conformity) marking. The VX1 software, without offering diagnostics, assists the practitioner in performing the diagnosis: based on dispersion measurements presented in the software, the practitioner can choose to mark zones of the heart to be subjected to catheter ablation.

In its current version, the VX1 software uses an annotated electrocardiogram database to train a machine learning engine that feeds back a value indicating the likelihood that a zone for which an electrocardiogram signal has been analysed is subject to atrial fibrillation. This is therefore not strictly speaking a measurement of the dispersion, nor is it information constituting a medical diagnosis, but an indication that there is a greater or lesser likelihood that an electrocardiogram evidences the occurrence of atrial fibrillation. Thus, the value fed back by the VX1 software is a value between 0 and 1 (0 indicating that there is only a near-zero probability that the zone being measured participates in fibrillation, and 1 that this probability is a near certainty). The software is configured such that a value of 0.5 is used as a threshold beyond which the attention of the practitioner is drawn to the potential presence of a dispersion.

During the development of the VX1 software, the Applicant has noticed that, in some cases, many zones of the heart feed back a value greater than 0.5, which complicates the work of the practitioner, since the latter must proceed with as little ablation as possible. In addition, when too many zones causing dispersion are identified, the practitioner tends to lose confidence in the measurement quality of the software, as it feeds back highly noisy information.

The invention improves the situation. To this end, it provides a device for processing spatiotemporal dispersion signals of electrocardiograms comprising a memory arranged to receive spatiotemporal dispersion signals of electrocardiograms associated on the one hand with a time marker and on the other hand with an electrocardiogram track, a calculator arranged to receive as input an electrogram track identifier and a time marker, to analyse the spatiotemporal dispersion signal of electrocardiograms associated with this electrogram track identifier, analyse a signal extract comprised between the time marker and the first previous time marker whose spatiotemporal dispersion signal value of electrocardiograms indicates a lack of dispersion, derive from this signal extract on the one hand a flatness value of the signal, and on the other hand a duration value derived from the duration of the signal extract, and feed back a track priority value calculated from the flatness value and the duration value, and a monitor arranged to receive the spatiotemporal dispersion signals of electrocardiograms associated with each electrocardiogram track, and, when the value of a spatiotemporal dispersion signal of electrocardiograms indicates a relevant dispersion, to call up the calculator with the corresponding time marker and electrogram track identifier.

This device is particularly advantageous because it makes it possible to offer additional information on the dispersion value which makes it possible to indicate to the practitioner a degree of priority of participation in fibrillation of the zone of the heart being the cause of the dispersion value fed back. Thus, the track priority value is information that allows the clinician to classify cardiac zones for ablation.

In addition, the track priority value will tend to increase when a practitioner stops on a given zone and this is the location of a dispersion. Thus, the track priority value allows the practitioner to perform their gesture more efficiently:
when a dispersion is detected, they know that it is desirable to stop on the zone concerned, and wait to see whether the track priority value increases. If this is not the case, they can be confident that it is desirable to continue their exploration.

According to various embodiments, the invention may have one or more of the following characteristics:
the spatiotemporal dispersion signals of electrocardiograms are sequences of values derived from electrogram signals each indicating a degree of confidence in the fact that a dispersion occurs for the considered electrogram track and time marker, and wherein the monitor and/or the calculator are arranged to determine whether a spatiotemporal dispersion signal of electrocardiograms indicates a relevant dispersion by comparing the value of the spatiotemporal dispersion signal of electrocardiograms to a threshold value,
the calculator is arranged to calculate the flatness value from at least one value among the standard deviation of the signal extract, the total variation of the signal extract, the entropy of the signal extract or a value derived from one or more derivatives of order greater than or equal to one of the signal extract,
the calculator is arranged to calculate the duration value by comparing the duration of the signal extract to a minimum value and/or to a maximum value, and feeding back the value 0 if the duration of the signal extract is less than the minimum value, feeding back the value 1 if the duration of the signal extract is greater than the maximum value, and otherwise feeding back a value between 0 and 1,
the calculator is arranged to determine the value between 0 and 1 by applying to the duration of the signal extract a projection function of the interval between the minimum value and the maximum value to the interval between 0 and 1, which projection function is chosen from the group comprising affine functions, exponential functions, polynomials, and threshold functions, and
the calculator receives spatiotemporal dispersion signals of electrocardiograms associated on the one hand with a time marker and on the other hand with an electrocardiogram track,
b) determining whether a value of a spatiotemporal dispersion signal of electrocardiograms indicates a dispersion,
c) if operation b) is negative, repeating it with a dispersion value having a subsequent time marker,
d) if operation b) is positive, analysing the spatiotemporal dispersion signal of electrocardiograms associated with the corresponding electrogram track identifier by analysing a signal extract comprised between the time marker and the first previous time marker whose spatiotemporal dispersion signal value of electrocardiograms indicates a lack of dispersion, and by deriving from this signal extract on the one hand a flatness value of the signal, and on the other hand a duration value derived from the duration of the signal extract, and
e) feed back a track priority value calculated from the flatness value and the duration value of operation d).

According to various embodiments, the method may have one or more of the following characteristics:
the spatiotemporal dispersion signals of electrocardiograms are sequences of values derived from electrogram signals each indicating a degree of confidence in the fact that a dispersion occurs for the considered electrogram track and time marker, and wherein operation b) comprises comparing a value of the spatiotemporal dispersion signal of electrocardiograms to a threshold value,
operation d) comprises calculating the flatness value from at least one value among the standard deviation of the signal extract, the total variation of the signal extract, the entropy of the signal extract or a value derived from one or more derivatives of order greater than or equal to one of the signal extract,
operation d) comprises calculating the duration value by comparing the duration of the signal extract to a minimum value and/or to a maximum value, and feeding back the value 0 if the duration of the signal extract is less than the minimum value, feeding back the value 1 if the duration of the signal extract is greater than the maximum value, and otherwise feeding back a value between 0 and 1,
operation d) comprises determining the value between 0 and 1 by applying to the duration of the signal extract a projection function of the interval between the minimum value and the maximum value to the interval between 0 and 1, which projection function being chosen from the group comprising affine functions, exponential functions, polynomials and threshold functions, and
operation e) comprises calculating the track priority value by performing a weighted, harmonic, weighted or arithmetic mean of the flatness value and the duration value.

The invention also relates to a computer program comprising instructions for executing the method according to the invention, a data storage medium o which such a computer program is recorded and a computer system comprising a processor coupled to a memory, the memory having recorded such a computer program.

### BRIEF DESCRIPTION OF THE DRAWINGS

Further characteristics and advantages of the invention will appear better upon reading the following description, with reference to examples given for illustrative and non-limiting purposes, with reference to the drawings wherein:

FIG. 1 shows a schematic diagram of a device according to the invention, and

FIG. 2 represents one example of implementation of an operating loop of the device of FIG. 1.

### DETAILED DESCRIPTION

The drawings and the description hereinafter essentially contain elements of certain nature. Hence, they could not only serve to better understand the present invention, but also contribute to the definition thereof, where appropriate.

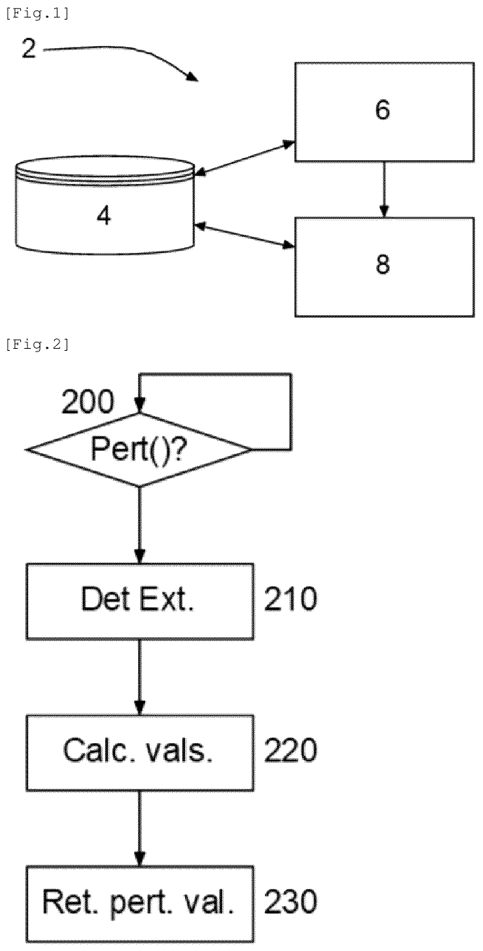

FIG. 1 represents a schematic example of a device 2 according to the invention. As has been specified in the introduction, signals that are used by the device are based on electrocardiograms measured by electrode pairs of a catheter in a patient's heart.

However, in the particular case of the invention, it is not these signals that are processed, but the dispersion measurement derived therefrom. As indicated in the introduction, the article by Seitz et al. “AF Ablation Guided by Spatiotemporal Electrogram Dispersion Without Pulmonary Vein Isolation: A Wholly Patient-Tailored Approach”, Journal of the American College of Cardiology, Volume 69, Issue 3, 24 Jan. 2017, pages 303-321 and the patent published under the number EP 3 236 843 allow better understanding what dispersion is both in terms of similarities and differences with CFAEs. Given the considered field and the fact that dispersion and CFAEs represent quite distinct phenomena insofar as CFAEs ignore any spatial aspect, these do not have a major interest in the context of the invention. Indeed, as will be seen below, the invention generally aims at qualifying stability of the signal formed by the dispersion values. This same analysis would only make little or no sense in the case of CFAEs.

The device 2 comprises a memory 4, a monitor 6 and a calculator 8.

The memory 4 is arranged to receive all the data, whether input or output, of a global or local nature, from the device 2. The memory 4 may consist of any data storage type capable of receiving digital data: hard drive, solid-state drive, flash memory in any form, random-access memory, magnetic disk, storage distributed locally or in the cloud, etc.

In the example described herein, the memory 4 receives all of the data regarding the device 2, i.e. the programs and software instantiating the monitor 6 and the calculator 8, the parameters and possible hyperparameters of these, the weights of the possible neural networks, the outputs and intermediate data of the neural networks, the spatiotemporal dispersion signals data of electrocardiograms received as input (where appropriate), the signal flatness and signal duration values, the data stored in buffer memory, as well as the output track priority value data. The data calculated by the device may be stored on any type of memory similar to the memory 4, or on the latter. These data may be erased after the device has performed its tasks or kept.

As will be seen below, the signal flatness value and the duration value are two values used to qualify whether the dispersion value signal is associated with an ablation-priority cardiac zone. These values are combined in order to produce a track priority value that indicates whether the dispersion value that is determined is associated with a dispersion that has significant potential to be associated with a zone that is the source of atrial fibrillation. Even more than in other patent applications filed by the Applicant, the track priority value does not constitute a diagnosis but an indication that allows a physician to make a decision, as would be blood pressure in another context.

The monitor 6 and the calculator 8 directly or indirectly access the memory 4. They could be made as an appropriate computer code executed on one or more processor(s). By processors, it should be understood any processor adapted to calculations described hereinbelow. Such a processor may be made in any known manner, in the form of a microprocessor for a personal computer, laptop, a tablet or a smartphone, an FPGA or SoC type dedicated chip, a computing resource on a grid or in the cloud, a cluster of graphical processing units (GPUS), a microcontroller, or any other form capable of providing the computing power necessary to the implementation described hereinbelow. One or more of these elements may also be made in the form of specialised electronic circuits such as an ASIC. A combination of a processor and of electronic circuits may also be contemplated. Machine-learning dedicated processors could also be contemplated.

The function of the monitor 6 is to analyse the data stream of spatiotemporal dispersion signals of electrocardiograms received as input, and to detect therein the fact that a dispersion value indicates that a treatment is necessary. As indicated in the introduction, in the context of the VX1 software, a dispersion value greater than or equal to 0.5 is significant. Of course, the detection of this value will depend on values taken by the dispersion signal received as an input. For example, this could be generated inversely to the VX1 software (for example 1—Value derived from VX1), in which case, it would rather be a value less than or equal to 0.5 that would be significant. This determination could also be carried out differently, based on a value derived from the derivative of the dispersion value signal, or in any other relevant manner.

Due to the continuous nature of processing by the device 2 which will appear better below, once a dispersion value has been detected by the monitor 6, the detection for the following values (but of course concerning the same track) may be different or simplified. Thus, in the case described above, rather than comparing the current dispersion value to a threshold, the monitor 6 may for example measure the derivative of the input dispersion value signal and consider that a detection is positive if the derivative is positive, etc. In general, the monitor 6 could rely on several tests in order to qualify the detection with a relevant dispersion value.

Hence, the role of the monitor 6 is of the “interruptive” nature. Indeed, in the absence of a relevant dispersion value, it is unnecessary to calculate a track priority value, since dispersion is not detected. On the other hand, as soon as a relevant dispersion value is detected, the monitor 6 calls up the calculator 8 to calculate the track priority value. Thus, the operation of the device 2 may appear as a detection loop by the monitor 6 for each track, with the execution of the calculator 8 each time a relevant dispersion value is detected. Of course, other implementations may be contemplated.

The role of the calculator 8 is to calculate the track priority value for a track whose dispersion value has been considered relevant by the monitor 6. As a reminder, the dispersion signal is a signal that associates a time marker with a dispersion value. This dispersion value itself is derived from an analysis of several electrocardiogram signal values. In the context of the VX1 software, the dispersion values are updated approximately every 300 ms, on the basis of extracts of electrocardiogram signals with a duration of approximately 1.5s. Alternatively, this update can take place every 100 ms, every 500 ms or the like.

As will be seen below, the determination of a track priority value is based on an extract of dispersion values which could have a continuous duration of 1.5 s, namely about 5 dispersion values, up to several dozen seconds, namely about one hundred dispersion values.

More precisely, the calculator 8 analyses each time a signal extract of dispersion values which ends with the dispersion value that has just been determined by the monitor 6 as relevant. This extract contains exclusively dispersion values including time markers that are successive, associated with the same track, and that are considered relevant by the monitor 6. It will appear that this extract can be obtained in many ways:
the monitor 6 can generate extracts during its operation, by adding a current dispersion value detected as relevant to a current extract if the immediately previous dispersion value has also been detected as relevant, or create a new extract if not,
the calculator 8, upon receiving a dispersion value associated with a given time marker, can analyse a buffer of past dispersion values, and stop at the oldest value considered relevant by the monitor 6, or even,
the calculator 8 can recover a buffer of past dispersion values from the time marker associated with a dispersion value received as an input, and cleanly determine an extract of this buffer that it considers relevant.

The work of the Applicant has demonstrated that the more accurate the dispersion value signal, the more useful the temporal continuity of relevant dispersion values. Indeed, with a dispersion considered as “noisy”, one could be tempted into ignoring an irrelevant dispersion value to have more data allowing calculating the track priority value. The work of the Applicant has demonstrated that the combination of the signal flatness value and the duration value makes it possible not to artificially enlarge size of the extracts and to obtain better results.

The calculator 8 operates performing two measurements on the extract defined above, a measurement of the flatness value of the signal, and a measurement of the duration value. In both cases, the aim is to determine whether the dispersion value has some stability over time. Indeed, the work of the Applicant revealed that signals with stable dispersion values were associated with priority zones in terms of ablation with a view to suppressing atrial fibrillation.

In the example described herein, the signal flatness value is derived from the standard deviation of the extract data. The work of the Applicant has revealed that the standard deviation is the measure providing the best results. Nonetheless, the Applicant has determined that other types of measures could be retained, such as variance, total variation of the extract, entropy of the extract, a value derived from one or more derivatives of order greater than or equal to one of the extract, extent, the interquartile range or another similar measure.

In parallel, the calculator 8 also determines a duration value which makes it possible to indicate how long the current extract is relative to a time interval considered to indicate that the cardiac zone associated with this dispersion has priority in terms of ablation. In the example described herein, the calculator 8 projects the duration of the extract over a standard interval between a minimum duration and a maximum duration. These two values, empirically estimated by the Applicant, indicate respectively the minimum duration that an extract should have to designate a priority cardiac zone, and the maximum duration given that the practitioner cannot afford to remain too long on each zone if they want to carry out their gesture within reasonable time frames and minimise the surgical risk.

In the example described herein, the calculator 8 determines the duration of the extract and projects its value over the interval [minimum duration; maximum duration] in order to determine a value between 0 and 1. The projection can be of any type: linear, polynomial, exponential, threshold, etc. This involves indicating, for a given extract duration, whether this duration is characteristic of a priority cardiac zone or not.

The minimum duration has an obvious usefulness as a floor value. The maximum duration has an important operational usefulness: when the first dispersion value of an extract longer than the maximum duration is detected, the priority value as an output will necessarily be low because the duration value will be low. As the extract grows, the track priority value will increase with the duration value. When an operator sees that the track priority value no longer moves, they can determine that it is because the duration value can no longer increase, and it is time to displace the catheter.

Alternatively, the calculator 8 could determine the duration value differently, independently of the range [minimum duration; maximum duration]. Still alternatively, the minimum duration and maximum duration could be variable during the procedure or be customised for each patient.

The flatness value of the signal and the duration value can be determined in parallel to each other. Alternatively, one can be calculated before the other.

The value between 0 and 1 is chosen because of how the calculator 8 determines the track priority value from the signal flatness value and the duration value. Indeed, the calculator 8 operates in the example described by operating a weighted harmonic mean. Alternatively, this mean may be harmonic, weighted or arithmetic for the flatness value and the duration value.

In the case where the signal flatness value and the duration value are not within identical value ranges, a relative adjustment may be performed, or another formula may be retained to calculate the track priority value.

FIG. 2 represents one example of an operating loop of the device 2. In an operation 200, the monitor 6 is called up with the current dispersion value for a given track. When the monitor 6 determines that a dispersion value is relevant, an operation 210 is triggered wherein the extract is determined from the time marker of the dispersion value of the operation 200, the associated track identifier, as well as the dispersion value signals already received for this track identifier. Once the extract has been determined, the calculator 8 can determine the flatness value and the duration value in an operation 220, then the track priority value and feed it back in an operation 230.

It appears that, if applicable, the device 2 will determine a track priority value per track for which a dispersion value signal is received as an input. This enriches the information transmitted to the practitioner to allow them to make the diagnosis determining whether a zone associated with a given track should be subjected to ablation or not. Please amend the claims as follow:

1. A device for processing spatiotemporal dispersion signals of electrocardiograms comprising:
a memory configured to receive spatiotemporal dispersion signals of electrocardiograms associated with a time marker and an electrocardiogram track,
a calculator configured to receive as input an electrogram track identifier and a time marker, to analyse the spatiotemporal dispersion signal of electrocardiograms associated with this electrogram track identifier, analyse a signal extract comprised between the time marker and the first previous time marker whose spatiotemporal dispersion signal value of electrocardiograms indicates a lack of dispersion, derive from this signal extract a flatness value of the signal, and a duration value derived from the duration of the signal extract, and feed back a track priority value calculated from the flatness value and the duration value, and
a monitor configured to receive the spatiotemporal dispersion signals of electrocardiograms associated with each electrocardiogram track, and, when the value of a spatiotemporal dispersion signal of electrocardiograms indicates a relevant dispersion, to call up the calculator with the corresponding time marker and electrogram track identifier.

2. The device of claim 1, wherein the spatiotemporal dispersion signals of electrocardiograms are sequences of values derived from electrogram signals each indicating a degree of confidence in the fact that a dispersion occurs for the considered electrogram track and time marker, and wherein the monitor and/or the calculator are configured to determine whether a spatiotemporal dispersion signal of electrocardiograms indicates a relevant dispersion by comparing the value of the spatiotemporal dispersion signal of electrocardiograms to a threshold value.

3. The device according to claim 1, wherein the calculator is configured to calculate the flatness value from at least one value from the standard deviation of the signal extract, the total variation of the signal extract, the entropy of the signal extract or a value derived from one or more derivatives of order greater than or equal to one of the signal extract.

4. The device according to claim 1, wherein the calculator is configured to calculate the duration value by comparing the duration of the signal extract to a minimum value and/or to a maximum value, and feeding back the value 0 if the duration of the signal extract is less than the minimum value, feeding back the value 1 if the duration of the signal extract is greater than the maximum value, and otherwise feeding back a value between 0 and 1.

5. The device according to claim 4, wherein the calculator is configured to determine the value between 0 and 1 by applying to the duration of the signal extract a projection function of the interval between the minimum value and the maximum value to the interval between 0 and 1, which projection function is chosen from the group comprising affine functions, exponential functions, polynomials, and threshold functions.

6. The device according claim 1, wherein the calculator is configured to calculate the track priority value by performing a weighted, harmonic, weighted or arithmetic mean of the flatness value and the duration value.

7. A method for determining a track priority value of a spatiotemporal dispersion signal of electrocardiograms comprising the following operations:
a) receiving spatiotemporal dispersion signals of electrocardiograms associated hand with a time marker and with an electrocardiogram track,
b) determining whether a value of a spatiotemporal dispersion signal of electrocardiograms indicates a dispersion,
c) if operation b) is negative, repeating it with a dispersion value having a subsequent time marker,
d) if operation b) is positive, analysing the spatiotemporal dispersion signal of electrocardiograms associated with the corresponding electrogram track identifier by analysing a signal extract comprised between the time marker and the first previous time marker whose spatiotemporal dispersion signal value of electrocardiograms indicates a lack of dispersion, and by deriving from this signal extract a flatness value of the signal, and a duration value derived from the duration of the signal extract, and
e) feed back a track priority value calculated from the flatness value and the duration value of operation d).

8. The method according to claim 7, wherein the spatiotemporal dispersion signals of electrocardiograms are sequences of values derived from electrogram signals each indicating a degree of confidence in the fact that a dispersion occurs for the considered electrogram track and time marker, and wherein operation b) comprises comparing a value of the spatiotemporal dispersion signal of electrocardiograms to a threshold value.

9. The method according to claim 7, wherein operation d) comprises calculating the flatness value from at least one value of the standard deviation of the signal extract, the total variation of the signal extract, the entropy of the signal extract or a value derived from one or more derivatives of order greater than or equal to one of the signal extract.

10. The method according to claim 7, wherein operation d) comprises calculating the duration value by comparing the duration of the signal extract to a minimum value and/or to a maximum value, and feeding back the value 0 if the duration of the signal extract is less than the minimum value, feeding back the value 1 if the duration of the signal extract is greater than the maximum value, and otherwise feeding back a value between 0 and 1.

11. The method according to claim 10, wherein operation d) comprises determining the value between 0 and 1 by applying to the duration of the signal extract a projection function of the interval between the minimum value and the maximum value towards the interval between 0 and 1, which projection function being chosen from the group comprising affine functions, exponential functions, polynomials and threshold functions.

12. The method according to claim 7, wherein operation e) comprises calculating the track priority value by performing a weighted, harmonic, weighted or arithmetic mean of the flatness value and the duration value.

13. A computer program comprising instructions to execute the method according to claim 7 when executed by a computer.

14. A data storage medium having the computer program according to claim 13 recorded thereon.
